(19) United States
(12) Patent Application Publication
(10) Pub. No.: US 2010/0210582 A1
(43) Pub. Date: Aug. 19, 2010

(54) METHOD AND COMPOSITION FOR TREATING PATIENTS WITH A HIGH RISK OF DEVELOPING NEURO-IMMUNE DISORDERS OR DEVELOPMENTAL DELAYS, IDENTIFIED THROUGH THE UTILIZATION OF LOW HOMOCYSTEINE OR ITS PRECURSORS/METABOLITES, AS THE PREDICTOR OF SUCH RISK

(76) Inventors: Kendal L. Stewart, Dripping Springs, TX (US); Stephen J. Orlando, Austin, TX (US)

Correspondence Address: ANDREWS KURTH LLP, 1350 I STREET, N.W., SUITE 1100, WASHINGTON, DC, 20005, US

(21) Appl. No.: 12/708,283

(22) Filed: Feb. 18, 2010

Related U.S. Application Data
(60) Provisional application No. 61/153,520, filed on Feb. 18, 2009.

(51) Int. Cl. A61K 31/519; A61K 31/7056; A61K 31/675; A61P 3/02; G01N 33/68

(52) U.S. Cl. 514/52; 514/249; 514/89; 436/89

(57) ABSTRACT

A method for predicting the likelihood of developing ASD and ASD-related diseases in a subject is disclosed. The method includes the steps of measuring the level of homocysteine, a homocysteine precursor or a homocysteine metabolite in the subject and evaluating the likelihood of developing ASD and ASD-related diseases based on the result of measurement. A subject is deemed to have a high risk of developing ASD and ASD-related diseases if the level of homocysteine, the homocysteine precursor or the homocysteine metabolite in the subject is at or below a threshold level. Also disclosed are a composition for reducing the risk of developing ASD, ASD-related diseases and adult neurological abnormalities and a method for determining a vaccination schedule in a subject.

### CROSS-REFERENCE TO RELATED APPLICATIONS

This application claims priority of U.S. Provisional Application Ser. No. 61/153,520, filed Feb. 18, 2009, which is incorporated by reference in its entirety.

### TECHNICAL FIELD

The technical field is medical diagnosis and treatment and, in particular, methods for predicting the risk of developing autism spectrum disorder and related diseases, as well as methods and compositions for reducing such a risk.

### BACKGROUND

Autism spectrum disorder (ASD) is a pervasive developmental disorder that causes severe and pervasive impairment in thinking, feeling, language, and the ability to relate to others. The onset is generally before the age of 3 years and is usually first diagnosed in early childhood. ASD can range from a severe form, called autistic disorder, through pervasive development disorder not otherwise specified (PDD-NOS), to much milder forms, such as attention deficit disorder/attention deficit hyperactivity disorder (ADD/ADHD), central auditory processing disorder (CAPD), learning disability (LD), Asperger syndrome, and atensi. ASD also includes two rare disorders, Rett syndrome and childhood disintegrative disorder. ASD has a prevalence of 0.6% in the population, affecting many more boys than girls.

The causes for ASD are complex. Many different disorders can result in ASD. Disorders such as the fragile X syndrome and tuberous sclerosis, which are both associated with autism, are inherited. Recent studies have found that the gene for at least one kind of familial autism may be on chromosome 13. Other factors, such as increased oxidative stress, aberrant immune response, impaired energy metabolism and environmental factors, may also lead to clinical symptoms and pathogenesis of ASD.

Early intervention has been shown to have a dramatic impact on reducing symptoms of ASD. Therefore, there is a need for methods of predicting the risk of developing ASD in children so that preventive and therapeutic measures may take place prior to or at the early stages of the disease development.

### SUMMARY

A method for predicting the likelihood of developing ASD and ASD-related diseases in a subject is disclosed. The method includes the steps of (a) measuring the level of homocysteine, a homocysteine precursor or a homocysteine metabolite in said subject and (b) evaluating the likelihood of developing ASD and ASD-related diseases based on the result of (a). The subject is deemed to have a high risk of developing ASD and ASD-related diseases if the level of homocysteine, the homocysteine precursor or the homocysteine metabolite in the subject is at or below a threshold level.

Also disclosed is a method for reducing the likelihood of developing ASD, ASD-related diseases and adult neurological abnormalities in a subject. The method includes measuring the level of homocysteine, a homocysteine precursor or a homocysteine metabolite in the subject; and, if the level of homocysteine, the homocysteine precursor or the homocysteine metabolite in said subject is at or below a threshold level, treating the subject with an effective amount of a pharmaceutical composition that comprises folinic acid and one or more compounds selected for the group consisting of hydroxy B-12, methyl B-12, and pyridoxal-5-phosphate and nicotinamide.

Also disclosed is a pharmaceutical composition for treating diseases relating to deficiencies of the methylation pathway. The pharmaceutical composition comprises folinic acid, one or more compounds selected from the group consisting of hydroxy B-12, methyl B-12, and pyridoxal-5-phosphate and nicotinamide, and a pharmaceutically acceptable carrier.

Also disclosed is a method for determining a vaccination schedule in a subject. The method includes: (a) measuring the level of homocysteine, a homocysteine precursor or a homocysteine metabolite in the subject and (b) determining the vaccination schedule for the subject based on the result of (a)

### DETAILED DESCRIPTION

One aspect of the present invention relates to a method for predicting the risk of developing ASD, ASD-related diseases and adult neurological abnormalities in a subject. In one embodiment, the method includes the steps of (a) measuring the level of homocysteine, a homocysteine precursor or a homocysteine metabolite in said subject; and (b) evaluating the likelihood of developing ASD, ASD-related diseases and adult neurological abnormalities based on the result of (a), wherein said subject is deemed to have a high risk of developing ASD, ASD-related diseases and adult neurological abnormalities, if the level of homocysteine, the homocysteine precursor or the homocysteine metabolite in said subject is at or below a threshold level.

The term “ASD” as used hereinafter, refers to pervasive developmental disorders that causes severe and pervasive impairment in thinking, feeling, language, and the ability to relate to others. ASD includes, but are not limited to, autistic disorder (AD), pervasive development disorder not otherwise specified (PDD-NOS), attention deficit disorder/attention deficit hyperactivity disorder (ADD/ADHD), central auditory processing disorder (CAPD), learning disability (LD), dyslexia, dysgraphia, Asperger syndrome and atensi.

The term “ASD-related diseases” as used hereinafter, refers to: developmental delays, speech delay, learning disorders, sensory integration disorders, and other neuro-immune disorders.

The term “adult neurological abnormalities” as used herein after, refers to: ADD/ADHD, Asperger syndrome, Guillian-Barre Syndrome, Multiple Sclerosis, Dementia, Vertigo, Lou Gehrig's disease, Parkinson disease, Adult Autistic Disorder, central auditory processing disorder (CAPD), learning disability (LD), dyslexia, dysgraphia, personality changes (Bi-Polar), psychosis, ataxia, and other neuro-immune disorders.

### Determination of Levels of Homocysteine, Its Precursors and Metabolites

Homocysteine is a compound with the formula HSCH₂CH₂CH(NH₂)CO₂H. It is a homologue of the naturally-occurring amino acid, cysteine, differing in that its side chain contains an additional methylene group. Homocysteine is typically derived from methionine by removing the latter's terminal Cε methyl group. Homocysteine is a product of the pathway known as the methionine cycle. It is so named because of the intermediates involved in the cycle and also because this is the cycle that is responsible for the process of methylation. The methionine cycle involves the regeneration of methionine from homocysteine. Briefly, methionine is converted to S-adenosylmethionine (SAM) by the enzyme methionine adenosyltransferase. SAM serves as a methyl-donor in many methyltransferase reactions and is converted to S-adenosylhomocysteine (SAH), which is then converted to homocysteine by adenosylhomocysteinase. This conversion of methionine to homocysteine occurs with the help of vitamin B12 (specifically the methyl version of vitamin B12, methylcobalamin) and folic acid (B9) in the 5-methyltetrahydrofolate form. Low levels of homocysteine and/or its metabolites are indications of deficiencies in the methylation pathway.

Methylation refers to the transfer of methyl groups from one chemical to another. Essentially any chemical compound that has a methyl group as part of its chemical structure is capable of donating the methyl group to another chemical that needs it. The chemical that receives the methyl group is “methylated”. This process of adding or removing methyl groups is necessary for the functioning of several biochemical reactions such as vitamin transport, DNA and RNA synthesis, creatinine generation, and immune responses involved in silencing infective vectors.

Numerous polymorphisms of the methylation enzymes have been identified. As a consequence of the biochemical reactions mentioned above, homocysteine is reduced in patients with poor methylation and can result in intra-cellular deficiencies of the necessary B vitamins. The relative deficiency of the methyl forms of vitamins folic acid (B₉), pyroxidine (B₆), or B12 (cyanocobalaomin) results in low homocysteine levels in quantification studies. While high plasma homocysteine is widely recognized as a cardiovascular disease risk factor, individuals with low homocysteine are at greater risk of immune deficiency states and therefore are at risk to handle oxidative stress poorly. The risk of poor response to oxidative stress is indicated by hypo-homocysteinemia because of homocysteine's role as a normal intermediate for conversion of methionine into cysteine, and ultimately for the production of glutathione, taurine and sulfate. Individuals with low homocysteine have limited capacity for response to oxidative stress, toxin exposure and poor T cell promotion. The immune deficiency state may also result in a variety of neuro-immune disorders such as ASD and ASD-related diseases.

Methods for determining the level of homocysteine in a biological sample (e.g., blood, urine and saliva) are well known in the art. Examples of such methods include chemical analysis methods (e.g., enzymatic assay or GC/MS) and immunological analysis (e.g., ELISA) methods. In certain embodiment, the homocysteine levels are determined using a plasma homocysteine assay such as fluorescence polarization immunoassay on the IMx® analyzer (Abbott Laboratories, Abbott Park, Ill.), a microtiter plate enzyme immunoassay (Bio-Rad Laboratories), or an HPLC kit with electrochemical detection (BAS).

The precursors of homocysteine include the intermediates or chemicals involved in the methionine cycle, such as S-adenosylmethionine (SAM or SAMe), S-adenosylhomocysteine (SAH) and methionine.

The metabolites of homocysteine include methionine, cystathionine, cysteine, cysteinylglycine, glycine, taurine, sulfate and glutathione.

Methods for determining the level of precursors and metabolites of homocysteine in a biological sample (e.g., blood, urine and saliva) are well known in the art. In certain embodiment, the levels of homocysteine precursors and/or metabolites are determined using plasma homocysteine assays, such as the fluorescence polarization immunoassay on the IMx® analyzer (Abbott Laboratories, Abbott Park, Ill.), the microtiter plate enzyme immunoassay (Bio-Rad Laboratories), or HPLC with electrochemical detection (BAS).

### Determination of the Likelihood of Developing ASD and ASD-Related Diseases Based on the Levels of Homocysteine, Homocysteine Precursors and Homocysteine Metabolites

A low homocysteine, homocysteine precursor or homocysteine metabolite level in a subject indicates a high risk of result of developing ASD and ASD-related diseases. A “low homocysteine level” refers to a homocysteine level at or below a threshold level. In one embodiment, the threshold level refers to a plasma homocysteine level in a range between 5.0 and 8.0 μMol/L (i.e., the threshold level may be defined as a level that is between 5.0 and 8.0 μMol/L, such as 6.75 μMol/L or 6.5 μMol/L). In another embodiment, the threshold level refers to a plasma homocysteine level in a range between 5.5 and 7.5 μMol/L. In another embodiment, the threshold level refers to a plasma homocysteine level in a range between 6.0 and 7.0 μMol/L. In yet another embodiment, the threshold level refers to a plasma homocysteine level of about 6.5 μMol/L.

In other embodiments, the threshold level refers to a urine homocysteine level in a range of 5.5 to 8.5 μMol/L, 6.0 to 8.0 μMol/L, or 6.5 to 7.5 μMol/L, In yet another embodiment, the threshold level refers to a urine homocysteine level of about 7.0 μMol/L.

In other embodiments, the threshold level refers to a saliva homocysteine level in a range of 5.5 to 8.5 μMol/L, 6.0 to 8.0 μMol/L, or 6.5 to 7.5 μMol/L, In yet another embodiment, the threshold level refers to a saliva homocysteine level of about 7.0 μMol/L.

The plasma, urine and saliva homocysteine threshold levels may further vary based on the age, sex and race of the subjects.

A “low homocysteine precursor level” or “low homocysteine metabolite level” refers to a level that is below the clinically acceptable normal range, or a level that is at or below a specifically defined threshold level. In certain embodiments, the threshold level for plasma methionine is about or at 26 μMol/L, the threshold level for plasma cystathionine is about or at 85 μMol/L, the threshold level for plasma cysteine is about or at 180 μMol/L, the threshold level for plasma cysteinylglycine is about or at 43 μMol/L, the threshold level for plasma glycine is about or at 230 μMol/L, the threshold level for plasma taurine is about or at 40 μMol/L, the threshold level for plasma sulfate is about or at 17 μMol/L, and the threshold level for plasma glutathione is about or at 10 μMol/L

A “high risk of developing ASD or ASD-related diseases” refers to a higher-than-normal probability to develop ASD or ASD-related diseases. The normal probability to develop ASD or ASD-related diseases may be determined from a statistically significant population.

In one embodiment, the risk evaluation in step (b) is performed in conjunction with one or more of other factors, including but are not limited to the subject's age, sex, race, ethnic background, genetic background, family history, medical history, xenobiotic exposures, and environmental exposures. In another embodiment, the risk evaluation step (b) is performed using a computer program that provides an overall evaluation of the likelihood of developing ASD or ASD-related disease using the result of (a) and one or more of other factors, including but are not limited to the subject's age, sex, race, ethnic background, genetic background, family history, medical history, xenobiotic exposures, and environmental exposures. In a related embodiment, the computer program includes a database containing threshold levels of homocysteine and/or homocysteine precursors/metabolites for different age, sex and race groups.

In another embodiment, the method of the present invention further includes the step of subjecting the subject at a high risk of developing ASD, ASD-related disease or adult neurological abnormalities to further medical examination. The medical evaluation may begin with a thorough medical history and physical examination, performed by a practitioner not only familiar with autism, but with other disorders that may appear similar to or mimic the symptoms of autism. The practitioner should have particular expertise in the neurological examination of impaired individuals, as subtle findings may lead the examiner down a particular diagnostic path.

The history and physical examination will point the examiner to specific diagnostic testing and/or objective diagnostic criteria to evaluate for other conditions associated with autism or developmental delay. For example, any child who has a language delay should have his or her hearing formally evaluated. Depending upon specific features of the examination and history, the practitioner may want to obtain blood and urine samples for specialized testing to evaluate for some of the inborn errors of metabolism and to obtain DNA for chromosomal studies and fragile X testing.

As used herein, “objective diagnostic criteria” refers to criteria capable of evaluation via the Autism Diagnostic Observation Schedule, the Bayley Scales of Infant Development, the Stanford-Binet Intelligence Scale, the Wechsler Preschool and Primary Scale of Intelligence, the Preschool Language Scale, the Receptive and Expressive One Word Vocabulary Tests, the Peabody Developmental Motor Scales for Gross and fine motor performance, the Assessment of Basic Language and Learning Skills, and/or a combination thereof. Other criteria capable of being measured and/or evaluated by other diagnostic tools or measures known and accepted in the art are also contemplated.

If the neurological examination is suggestive of a structural brain lesion, then a neuroimaging study, such as a brain CT (“CAT scan”) or MRI scan, may be performed.

### Homocysteine Levels and Vaccination

Studies have demonstrated that homocysteine is a potent concentration-dependent T cell activator promoting cellular activation and differentiation. Homocysteine appears to exert diverse effects on immune function in the circulation and within the intracellular and tissue microenvironment. Low homocysteine levels in the body do not allow adequate T cell activation and/or development and induces a state of T cell lymphocyte deficiency and/or dysfunction. The lymphocyte deficient state and/or dysfunction produces the potential for poor response to vaccinations.

Moreover, vaccinations by nature induce an immune response by introducing immune recognized material (antigen) that is specific for the infection for which you wish to produce immunity towards. The introduction of antigenic material produces oxidative stress (inflammation) from the immune response. The more antigen introduced at one time, the more oxidative stress produced in the body. Multiple antigen vaccinations (e.g., DTaP) and the live, attenuated viral vaccinations (e.g., Measles, Mumps and Rubella (MMP), Varicella) induce the most aggressive immunological response and therefore, the most aggressive oxidative stress. In children possessing low levels of homocysteine, as a result of genetic polymorphisms or by other means, the introduction of an aggressive vaccination schedule recommended by the American Academy of Pediatrics and the Center for Disease Control can increase the risk of development of potential disease states (e.g., autism, autistic spectrum disorders and related maladies) in these patients. Overall, a low homocysteine level and resultant T cell immune dysfunction in a child indicate a need to reevaluate the standard recommendations for childhood vaccinations due to the potential risk of subsequent disorder development, and to amend, delay, or suspend the standard vaccination schedule if necessary.

Therefore, another aspect of the present invention relates to a method for determining the vaccination schedule of a subject. The method include the steps of (a) measuring the level of homocysteine, a homocysteine precursor or a homocysteine metabolite in the subject and (b) determining the vaccination schedule for the child based on the result of (a).

In certain embodiments, multi-antigen and live viral vaccinations are deferred until normalization of homocysteine levels in the subject with a low plasma homocysteine level. In another embodiment, multi-antigen and live viral vaccinations are deferred until the age of normal development of the subject's sensory nervous system. The development of sensory nervous system includes sensory integration of the subject's proprioceptive sense, visual sense and vestibular system, typically referenced between the ages of 2 years and 3 years. In another embodiment, multi-antigen and live viral vaccinations are deferred until the subject has been treated for methylation deficiency for a specified time period. The length of the treatment period is determined by the health care provider and can be, for example, 3, 6, 9 or 12 months.

In other embodiments, the determination of the vaccination schedule is made based on the homocysteine level and other clinical considerations, such as the immune status and/or health condition of the subject, the likelihood of contracting the diseases to be immunized against, the family/medical history of the subject, and potential xenobiotic and environmental exposures.

### Treatment of Deficiencies of Methylation Pathway

As noted above, low levels of homocysteine, homocysteine precursors or homocysteine metabolites are indications of deficiencies in the methylation pathway. Such deficiencies include folic acid deficiency and vitamin B12 deficiency, both of which have been independently associated with decreased immune function, the apoptosis (death) of bone marrow hematopoietic progenitor cells and the appearance of leukocytes with hypo-methylated DNA in the peripheral circulation.

Therefore, another aspect of the present invention relates to a method for treating a subject with deficiencies of methylation pathway, which are often manifested by having low levels of homocysteine, homocysteine precursors or homocysteine metabolites. In certain embodiments, the method includes the step of administering to a subject having a low level of homocysteine, homocysteine precursors or homocysteine metabolites an effective amount of a pharmaceutical composition comprising folinic acid and one or more compounds selected from the group consisting of methyl B-12, hydroxy B-12, nicotinamide and pyridoxal-5-phospate (P-5-P).

In one embodiment, the one or more compounds include methyl B-12. In another embodiment, the one or more compounds include methyl B-12 and pyridoxal-5-phosphate. In another embodiment, the one or more compounds include methyl B-12, pyridoxal-5-phosphate and nicotinamide. In yet another embodiment, the one or more compounds include hydroxy B-12, methyl B-12, pyridoxal-5-phosphate and nicotinamide.

The pharmaceutical composition may be administered orally, sublingually, transdermally, or by injections. The term “effective amount” or “therapeutically effective amount” means an amount effective, when administered to a patient, to provide any therapeutic benefit. A therapeutic benefit may be an elevation of homocysteine level, homocysteine metabolite levels or homocysteine precursor levels in the patient receiving the treatment, an amelioration of symptoms of ASD, ASD-related diseases, and adult neurological abnormalities or a delay or postponement of the development of ASD, ASD-related diseases and adult neurological abnormalities. In certain circumstances a patient may not present symptoms of a condition for which the patient is being treated. A therapeutically effective amount of an active ingredient may also be an amount sufficient to provide a significant positive effect on any indicium of a disease, disorder, or condition, e.g. an amount sufficient to significantly reduce the frequency and severity of symptoms. A significant effect on an indicium of a disease, disorder, or condition is statistically significant in a standard parametric test of statistical significance, for example Student's T-test, where p≦0.05. An “effective amount” or “therapeutically effective amount” of the B vitamin formulations provided herein may be an amount of any dosage amount approved by a governmental authority such as the U.S. FDA, for use in the treatment.

In some embodiments, the pharmaceutical composition is formulated for oral administration and comprises one or more of the following per dosage form:

1) folinic acid with a range between 0.5 mg and 10 mg and more preferably about 10 mg, and still more preferably is 10 mg;

2) hydroxy B-12 with a range between 5 mg and 12.5 mg and more preferably about 6.25 mg, and still more preferably is 6.25 mg;

3) methyl B-12 with a range between 5 mg and 12.5 mg and more preferably about 6.25 mg, and still more preferably is 6.25 mg;

4) pyridoxal-5-phosphate with a range between 5 mg and 25 mg and more preferably about 6.25 mg, and still more preferably is 6.25 mg; and

5) nicotinamide with a range between 1 mg and 10 mg and more preferably about 1.0 mg and still more preferably is 1.0 mg.

In other embodiments, the pharmaceutical composition is formulated for sublingual administration and comprises one or more of the following per dosage form:

1) folinic acid with a range between 0.5 mg and 10 mg and more preferably about 5 mg, and still more preferably is 5 mg;

2) hydroxy B-12 with a range between 5 mg and 12.5 mg and more preferably about 6.25 mg, and still more preferably is 6.25 mg;

3) methyl B-12 with a range between 5 mg and 12.5 mg and more preferably about 6.25 mg, and still more preferably is 6.25 mg;

4) pyridoxal-5-phosphate with a range between 5 mg and 12.5 mg and more preferably about 6.25 mg, and still more preferably is 6.25 mg; and

5) nicotinamide with a range between 0.5 mg and 10 mg and more preferably about 1.0 mg and still more preferably is 1.0 mg.

In other embodiments, the pharmaceutical composition is formulated for injectable administration and comprises one or more of the following per dosage form:

1) folinic acid with a range between 0.5 mg and 5 mg and more preferably about 3 mg, and still more preferably is 3 mg;

2) hydroxy B-12 with a range between 5 mg and 12.5 mg and more preferably about 6.25 mg, and still more preferably is 6.25 mg;

3) methyl B-12 with a range between 5 mg and 12.5 mg and more preferably about 6.25 mg, and still more preferably is 6.25 mg;

4) pyridoxal-5-phosphate with a range between 5 mg and 12.5 mg and more preferably about 6.25 mg, and still more preferably is 6.25 mg; and

5) nicotinamide with a range between 0.5 mg and 10 mg and more preferably about 1.0 mg and still more preferably is 1.0 mg.

In other embodiments, the pharmaceutical composition is formulated for transdermal administration and comprises one or more of the following per dosage form:

1) folinic acid with a range between 0.5 mg and 5 mg and more preferably about 5 mg, and still more preferably is 5 mg;

2) hydroxy B-12 with a range between 5 mg and 12.5 mg and more preferably about 6.25 mg, and still more preferably is 6.25 mg;

3) methyl B-12 with a range between 5 mg and 12.5 mg and more preferably about 6.25 mg, and still more preferably is 6.25 mg;

4) pyridoxal-5-phosphate with a range between 5 mg and 12.5 mg and more preferably about 6.25 mg, and still more preferably is 6.25 mg; and

5) nicotinamide with a range between 0.5 mg and 10 mg and more preferably about 1.0 mg and still more preferably is 1.0 mg.

The dosage of the pharmaceutical composition that is administered is based on the patient's weight and is usually administered every other day, but can be administered daily at reduced dosages for a short period of time.

In another embodiment, the method includes the steps of (a) measuring the level of homocysteine, a homocysteine precursor or a homocysteine metabolite in a subject, (b) if the subject has a low level of homocysteine, the homocysteine precursor, or the homocysteine metabolite, treating the subject with an effective amount of a pharmaceutical composition comprising one or more active ingredients selected from the group consisting of folinic acid, methyl B-12, hydroxy B-12, nicotinamide and pyridoxal-5-phospate (P-5-P) for a desired period of time, and (c) deferring multi-antigen and/or live viral vaccinations in the subject.

In one embodiment, the multi-antigen and live viral vaccinations are deferred until the level of homocysteine exceeds the threshold level in the subject.

In another embodiment, the multi-antigen and live viral vaccinations are deferred until the subject reaches the age of normal development of the sensory nervous system.

In another embodiment, the multi-antigen and live viral vaccinations are deferred until the completion of step (b).

Examples of the live viral vaccinations include, but are not limited to, Varicella vaccine, measles vaccine, mumps vaccine and rubella vaccine.

In one embodiment, the desired period of time is about 1 months, about 2 months, about 3 months, about 6 months, about 9 months, or about 12 months as determined by the health care provider. In another embodiment, the desired period of time is the time period required to restore the homocysteine level in the subject to normal levels using the treatment of step (b).

In another embodiment, the method includes, the steps of (a) measuring the level of homocysteine, a homocysteine precursor or a homocysteine metabolite in a subject, (b) if the subject has a low level of homocysteine, the homocysteine precursor, or the homocysteine metabolite, treating the subject with an effective amount of a pharmaceutical composition comprising one or more active ingredients selected from the group consisting of folinic acid, methyl B-12, hydroxy B-12, nicotinamide and pyridoxal-5-phospate (P-5-P) for a desired period of time, and (c) deferring multi-antigen and/or live viral vaccinations in the subject until after the completion of step (b).

In yet another embodiment, the method includes the steps of (a) measuring the level of homocysteine or a metabolite of homocysteine in the subject, (b) determining the likelihood of developing ASD and ASD-related diseases based on the result of (a), and (c) if the subject is determined to have a high risk of developing ASD and ASD-related disease, treating the subject with an effective amount of a pharmaceutical composition comprising one or more active ingredients selected from the group consisting of folinic acid, methyl B-12, hydroxy B-12, nicotinamide and pyridoxal-5-phospate (P-5-P) for a desired period of time as determined by the health care provider.

In another embodiment, a subject is deemed to have a high risk of developing ASD and ASD-related disease, if the subject has a plasma homocysteine level that is at or below a threshold level. In one embodiment, the threshold level refers to a plasma homocysteine level in a range between 5.0 and 8.0 μMol/L (i.e., the threshold level may be defined as a level that is between 5.0 and 8.0 μMol/L, such as 6.75 μMol/L or 6.5 μMol/L). In another embodiment, the threshold level refers to a plasma homocysteine level in a range between 5.5 and 7.5 μMol/L. In another embodiment, the threshold level refers to a plasma homocysteine level in a range between 6.0 and 7.0 μMol/L. In yet another embodiment, the threshold level refers to a plasma homocysteine level of about 6.5 μMol/L.

Also disclosed is a method for ameliorating symptoms associated with a neurological sequelae in a subject. The method comprises administering to the subject an effective amount of a pharmaceutical composition comprising: folinic acid; and one or more compounds selected from the group consisting of hydroxy B-12, methyl B-12, and pyridoxal-5-phosphate and nicotinamide, wherein said neurological sequelae includes ASD, ASD-related diseases and adult neurological abnormalities.

### Pharmaceutical Compositions

Another aspect of the present invention relates to a pharmaceutical composition comprising a pharmaceutically acceptable carrier, folinic acid, and one or more compounds selected from the group consisting of methyl B-12, hydroxy B-12, nicotinamide and pyridoxal-5-phospate (P-5-P). The pharmaceutical composition can be formulated as described herein.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The pharmaceutical composition of the invention is formulated to be compatible with its intended route of administration. Examples of routes of administration include, oral, sublingual, transdermal (e.g., topical), parenteral (e.g., intravenous, intra-arterial, intradermal, subcutaneous, intramusclar, intra-peritoneal), and transmucosal, 1 administration.

Pharmaceutical compositions suitable for oral or sublingual administration generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediamine tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Sterile injectable solutions can be prepared by incorporating the active compound (e.g., folinic acid, methyl B-12, hydroxy B-12, nicotinamide and/or pyridoxal-5-phospate (P-5-P).) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

In one embodiment, the pharmaceutical compositions are prepared with carriers that will protect the active ingredients against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is especially advantageous to formulate oral or parenteral compositions in dosage unit form for ease of administration and uniformity of dosage. Dosage unit form, as used herein, includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

### Examples

### Example 1

### Plasma Homocysteine Levels in Children with ASD or ASD-Related Diseases

Plasma homocysteine levels were determined in three hundred and eighty-three children with ASD or ASD-related diseases. As shown in Table 1, the plasma homocysteine levels in 87.7% of the children were below 6.5 μMol/L, indicative of a deficient methylation pathway and/or an immune system deficiency. The study confirmed that a low plasma homocysteine level may be used as an indicator for the likelihood of developing ASD or ASD-related diseases.

TABLE 1
Plasma homocysteine levels in autistic children

| | TOTAL |
|---|---|
| Total Data Points | 383 |
| At 6.75 Value | |
| Above | 42 |
| | 11.0% |
| Below | 341 |
| | 89.0% |
| at 6.50 Value | |
| Above | 47 |
| | 12.3% |
| Below | 336 |
| | 87.7% |

### Example 2

### Plasma Homocysteine Levels in Adults Suffering from Neurosensory Disorders

Plasma homocysteine levels were determined in adults suffering from neurosensory disorders. As shown in Table 2, low homocysteine levels were predominant in adult patients suffering from neurosensory disorders.

TABLE 2
Plasma homocysteine levels in adults with neurosensory disorders.

| | |
|---|---|
| Adults | 11 |
| at 6.50 Value | |
| Above | 4 |
| | 36.4% |
| Below | 7 |
| | 63.6% |

### Example 3

### Treatment of Patients with ASD, ASD-Related Diseases, and Adult Neurological Abnormalities with Methyl B Formulation

Patients with ASD, ASD-related diseases, or adult neurological abnormalities were treated with the following Methyl B formulations:

Formulation 1: folinic acid (20 mg), methyl B-12 (12.5 mg), pyridoxal-5-phospate (P-5-P) (12.5 mg) and nicotinamide (1.0 mg) in a 250 mg/ml capsule, administered orally every other day;

Formulation 2: folinic acid (20 mg), methyl B-12 (12.5 mg), pyridoxal-5-phospate (P-5-P) (12.5 mg) and nicotinamide (1.0 mg) in a 500 mg/ml capsule, administered every other day;

Formulation 3: folinic acid (3 mg), methyl B-12 (12.5 mg) and pyridoxal-5-phospate (P-5-P) (6.25 mg) in a 5 ml vial, administered by injection every other day in a dosage as defined below based on the patient's weight;

Formulation 4: folinic acid (3 mg), methyl B-12 (6.25 mg), hydroxyl B-12 (6.25 mg) and pyridoxal-5-phospate (P-5-P) (6.25 mg) in a 5 ml vial, administered by injection every other day in a dosage as defined below based on the patient's weight;

Formulation 5: folinic acid (3 mg), methyl B-12 (12.5 mg) and hydroxyl B-12 (6.25 mg) in a 5 ml vial, administered by injection every other day in a dosage as defined below based on the patient's weight; and

Formulation 6: folinic acid (5 mg), methyl B-12 (12.5 mg), hydroxyl B-12 (6.25 mg) and pyridoxal-5-phospate (P-5-P) (12.5 mg) in a cream, administered topically in an amount of 1-2 ml per day.

Injection dosages that are applied every other day are based on weight as follows:

1) 0.1 cc if weight between 0 and 65 lbs;

2) 0.15 cc if weight is between 65 and 85 lbs;

3) 0.2 cc of weight is between 85 and 125 lbs;

4) 0.3 cc if weight is between 125 and 170 lbs; and

5) 0.4 cc if weight is over 170 lbs.

The initial results indicate that the formulation containing folinic acid and methyl B-vitamins provides much better response in patients than formulations with methyl B-vitamins only. Patient improvements were indicated through personal/parental assessment of patient's health status, objective clinical assessments, and a higher homocysteine level on the majority of patients whose homocysteine level was retested.

The embodiments and examples described above are intended to be illustrative and not limiting. It should be understood that modifications and variations can be made by persons skilled in the art in light of the above teachings. Therefore, changes may be made in the particular embodiments disclosed which are within the scope of what is described as defined by the appended claims.

What is claimed is:

1. A pharmaceutical composition for treating deficiencies of the methylation pathway, comprising:
folinic acid;
one or more compounds selected from the group consisting of hydroxy B-12, methyl B-12, and pyridoxal-5-phosphate and nicotinamide; and
a pharmaceutically acceptable carrier.

2. The pharmaceutical composition of claim 1, wherein said one or more compounds is folinic acid.

3. The pharmaceutical composition of claim 1, wherein said one or more compounds are methyl B-12 and pyridoxal-5-phosphate.

4. The pharmaceutical composition of claim 1, wherein said one or more compounds are methyl B-12, pyridoxal-5-phosphate and nicotinamide.

5. The pharmaceutical composition of claim 1, wherein said one or more compounds are hydroxy B-12, methyl B-12, pyridoxal-5-phosphate and nicotinamide.

6. A method for treating a deficiency of the methylation pathway in a subject, comprising:
administrating to the subject an effective amount of the pharmaceutical composition of claim 1.

7. The method of claim 6, wherein said pharmaceutical composition is administered orally.

8. The method of claim 6, wherein said pharmaceutical composition is administered sublingually.

9. The method of claim 6, wherein said pharmaceutical composition is administered by injection.

10. The method of claim 6, wherein said pharmaceutical composition is administered transdermally.

11. A method for predicting the likelihood of developing ASD, ASD-related diseases and adult neurological abnormalities in a subject, comprising:
(a) measuring the level of homocysteine, a homocysteine precursor or a homocysteine metabolite in said subject; and
(b) evaluating the likelihood of developing ASD, ASD-related diseases and adult neurological abnormalities based on the result of (a),
wherein said subject is deemed to have a high risk of developing ASD, ASD-related diseases and adult neurological abnormalities if the level of homocysteine, the homocysteine precursor or the homocysteine metabolite in said subject is at or below a threshold level.

12. The method of claim 11, wherein the likelihood of developing ASD, ASD-related diseases and adult neurological abnormalities is evaluated based on the result of (a) and one or more factors selected from the group consisting of the subject's age, the subject's sex, the subject's ethnic background, the subject's genetic background, the subject's family history, the subject's medical history, the subject's xenobiotic exposure, and the subject's environmental exposure.

13. The method of claim 11, wherein said subject is deemed to have a high risk of developing ASD, ASD-related diseases and adult neurological abnormalities if the level of plasma homocysteine is at or below a threshold that lies within a range of between 5.0 and 8.0 uMol/L.

14. The method of claim 11, wherein said subject is deemed to have a high risk of developing ASD, ASD-related diseases and adult neurological abnormalities if the level of plasma homocysteine is at or below a threshold of about 6.5 uMol/L.

15. A method for reducing the likelihood of developing ASD, ASD-related diseases and adult neurological abnormalities in a subject, comprising:
measuring the level of homocysteine, a homocysteine precursor or a homocysteine metabolite in said subject; and
if the level of homocysteine, the homocysteine precursor or the homocysteine metabolite in said subject is at or below a threshold level, treating the subject with an effective amount of a pharmaceutical composition that comprises folinic acid and one or more compounds selected for the group consisting of hydroxy B-12, methyl B-12, and pyridoxal-5-phosphate and nicotinamide.

16. The method of claim 15, further comprising:
deferring multi-antigen and live viral vaccinations for said subject.

17. The method of claim 16, wherein said multi-antigen and live viral vaccinations are deferred until the level of homocysteine, homocysteine precursor or homocysteine metabolite exceeds the threshold level in said subject.

18. The method of claim 16, wherein said multi-antigen and live viral vaccinations are deferred until the subject reaches the age of normal development of the sensory nervous system.

19. A method for determining a vaccination schedule in a subject, comprising:
(a) measuring the level of homocysteine, a homocysteine precursor or a homocysteine metabolite in said subject;
(b) determining the vaccination schedule for said subject based on the result of (a)

20. The method of claim 19, further comprising:
deferring multi-antigen and live viral vaccinations for said subject if the level of homocysteine, the homocysteine precursor or the homocysteine metabolite in said subject is at or below a threshold level.
